(19) United States
(12) Patent Application Publication
(10) Pub. No.: US 2002/0068881 A1
(43) Pub. Date: Jun. 6, 2002

(54) Cervical cytology instrument

(76) Inventors: Myles S. Kobren, Woodbury, NY (US); Andrew W. Menzin, Roslyn Heights, NY (US)

Correspondence Address: Eliot S. Gerber, Fay, Kaplun & Marcin LLP, 100 Maiden Lane, New York, NY, 10038, US

(21) Appl. No.: 09/728,987

(22) Filed: Dec. 4, 2000

(51) Int. Cl. A61B 10/00

(52) U.S. Cl. 600/569

(57) ABSTRACT

A gynecological instrument for collecting cell samples from the endocervix and exocervix and exocervix, e.g., a Pap Smear brush, has a handle portion carrying a brush base. The brush is connected to the brush base and comprises a central portion of stiff bristles which are generally perpendicular to the handle axis, and a line of softer bristles generally aligned with that axis.

### FIELD OF THE INVENTION

The present invention relates to medical devices and more particularly to gynecological instruments for collecting cell samples from the endocervix and exocervix, e.g., “Pap Smear” brushes.

### BACKGROUND OF THE INVENTION

At the present time, the “Pap smear” (Papanicolou Smear) is one of the most important procedures in gynecology and one of the most effective cancer screening tests in history. It is non-invasive and without risk and provides many benefits to patients. If the cells are adequately collected and correctly analyzed, the Pap smear can detect cancers and pre-cancers of the lower genital wall, i.e., cancer of the cervix. This has been demonstrated to have led to a marked reduction in the incidence of cervical cancer and improved survival, which occurs because the cancers are caught early. Other treatable medical conditions may also be detected on a Pap smear. New technology has also allowed the coupling of other important medical tests (such as STD testing) to the material obtained for Pap smear examination.

Various devices are commonly used to obtain a Pap smear. For example, a cotton swab at the end of a stick may be used to obtain cells from the outer surface of the cervix, that type of device being called a cervical swab smear. Another device, to obtain cells from the surface of the cervix (exocervical or ectocervical) and from the endocervix (cervical canal), is a wooden or plastic spatula (stick) having a flat broad edge and two lobes. However, such a spatula may not obtain a sufficient amount of vaginal wall cells. To obtain endocervical cells, it is common to use an endocervical aspirator or an endocervical brush having bristles at the end of a metal wire, like a pipe-cleaner brush. In addition, a widely used device to obtain both types of cells is a plastic brush called a “Pap broom” or a “Papette” (TM of Wallach Surgical Devices) having elongated vertical plastic bristles arranged in a fan shape. Such devices may, though, not penetrate the endocervical canal and retrieve sufficient endocervical cells.

Pap smears may be prepared in a conventional fashion using a glass slide or with a liquid based technique. The smears are then examined by a cytotechnologist and a pathologist.

In a significant number of instances, the specimens are not adequate, often because of lack of endocervical cells, e.g., the endocervical cell sample is insufficient for the test. When a patient is called for a re-examination, because of the inadequacy of the sample, she may experience anxiety, lose time from work and perhaps undergo needless extra examinations, as well as be unduly alarmed.

The prior patents listed below, and incorporated by reference, show that many attempts have been made to provide a satisfactory cervical sampling brush.

U.S. Pat. No. 4,754,764 shows a brush having a central extending portion and a flat disk portion. It also shows, at FIG. 1, the prior art spatula and endocervical brush.

In U.S. Pat. Nos. 4,762,133 and 4,873,992 one pipe-cleaner is perpendicular to another pipe-cleaner type brush.

### SUMMARY OF THE INVENTION

In accordance with the present invention, there is provided a Pap smear brush whose purpose is to simultaneously collect a sufficient amount of exocervix and endocervical and vaginal cells for pathological evaluation. The brush is low in cost, disposable, and will not injure, or unnecessarily discomfort, the patient.

The brush of the present invention is a fan-like arrangement of soft plastic filaments, a flexible and bendable handle portion which will retain the shape into which it is bent, and a central brush portion having relatively stiff bristles. Those stiff bristles are arranged generally perpendicular to the axis of the handle. The softer and more flexible bristles are arranged generally parallel (within 40°) of the imaginary central axis of the handle portion. The handle portion is bendable, preferably at two places, to accommodate patients whose cervixes are acutely bent, so that a straight non-bendable instrument could not be used.

In the first embodiment the central brush portion does not extend, or extends only slightly, from the fan-shaped brush portion. The bristles (filaments) of the fan-shaped portion are angled outwardly from the handle axis at about 20°.

The second embodiment is the same as the first embodiment, except that the central brush portion extends beyond the fan-shaped bristles.

In the third embodiment the fan-shaped bristles are aligned parallel to the handle axis, and are not angled outwardly.

### BRIEF DESCRIPTION OF THE DRAWINGS

The following detailed description of the invention should be taken in conjunction with the accompanying drawings.

In the drawings:

FIG. 1 is a front plan view of the Pap smear brush of the first embodiment;

FIG. 2 is a side plan view of the embodiment of FIG. 1;

FIG. 3 is a front plan view of the brush of the second embodiment; and

FIG. 4 is a side plan view of the brush of the second embodiment.

### DETAILED DESCRIPTION OF THE INVENTION

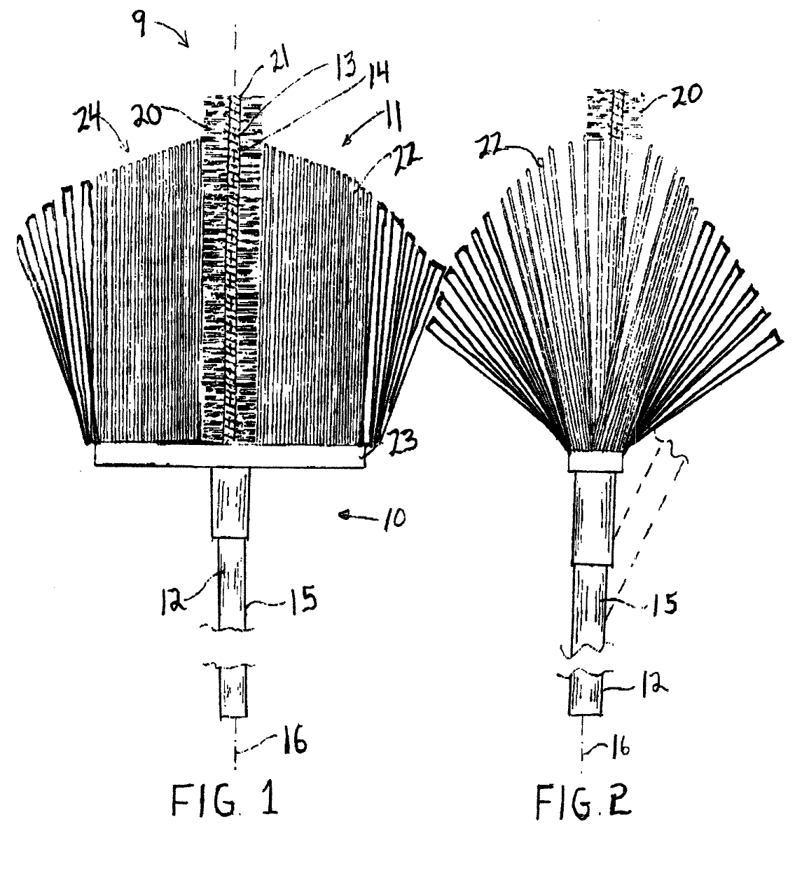

As shown in FIG. 1, the first embodiment of the Pap smear brush 9 of the present invention consists of a handle portion 10 and a brush portion 11 proximate the distal end of the handle portion 10. The handle portion 10 has a handle 12 which preferably consists of a plastic shaft or of two wires 13,14 which are twisted about each other. See FIG. 1 for a view of the wires 13, 14. These wires are similar to those of a pipe cleaner. The handle 12 is bendable and will remain in the general position to which it is bent. For example, as shown in FIG. 2, the handle 12 may be bent at bend 15 to a 30° angle relative to the imaginary central axis 16 of the handle 12.

The brush portion 11 consists of two types of bristles. The endocervical bristles 20 of the central arm 21 are plastic and are relatively stiff and arranged generally perpendicular to axis 16. They are held by the twisted wires 13, 14. They are similar to the bristles of a pipe-cleaner and are preferably 1-2 mm in length.

The exocervical fan bristles 22 are plastic, and softer and more flexible than bristles 20. The fan bristles 22 extend upwardly (as seen in FIGS. 1 and 2) from their base 23, the fan bristles 22 and base 23 being integrally formed, preferably by injection molding. As seen in FIG. 1, the fan bristles form a fan-like arrangement 24. The central arm bristles 20 extend above the level of the softer bristles 22. As seen in FIG. 2, the bristles extend outwardly from axis 16, preferably in the range of 10° to 40° outwardly.

In operation, the central arm 21 is placed within the endocervical canal of the patient and anchors the brush 9 so that it may be rotated (about its axis 16). The fan bristles 22, during such rotation, swab the endocervix and the vagina. The brush 9 will collect a sufficient sample of endocervical, ectocervical and vaginal walls for pathological evaluation. This instrument is valuable for both pre- and post-menopausal patients as it samples tissue to evaluate a large surface area of the vagina.

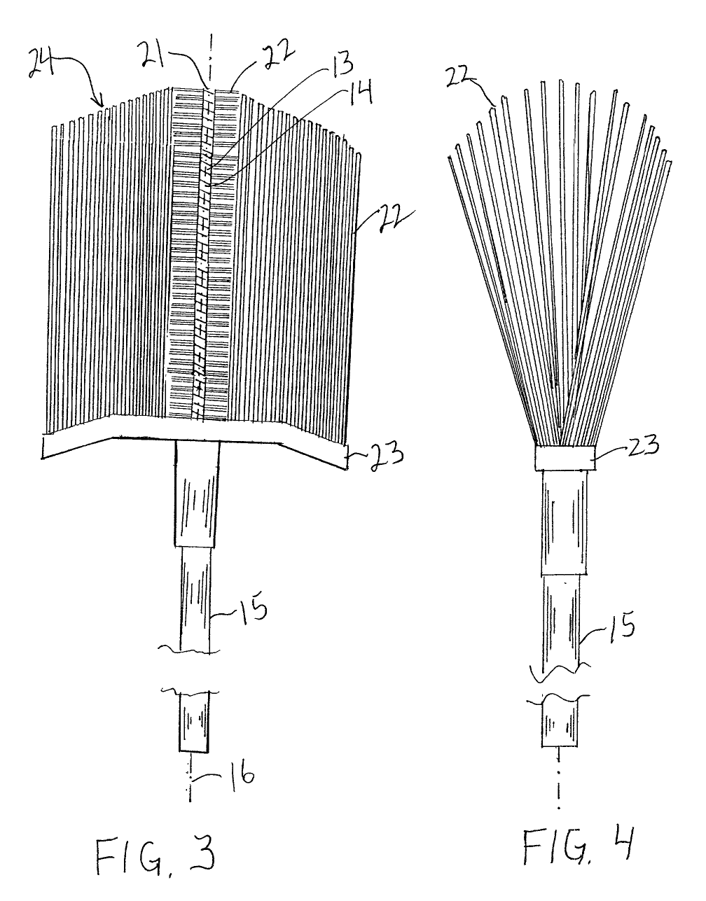

In the second embodiment of FIGS. 3 and 4, the stiff bristles 20 of the central arm are not higher (above) the level of the softer (more flexible) bristles 22. As in the prior embodiments, the bristles are fan-shaped (front view of FIG. 3), are arranged on a base 23, and are angled outwardly from the central axis (side view of FIG. 4).

What is claimed is:

1. A medical Pap smear brush for the simultaneous collection of both exocervical and endocervical cells from a patient, the brush comprising:
(a) an elongated handle portion having proximal and distal ends and an imaginary axis;
(b) a brush base mounted on the handle portion and a plurality of relatively soft and flexible bristles, said flexible bristles being aligned generally (within 40°) parallel to said axis and aligned in a line and not protruding from a disk;
(c) a plurality of relatively stiff bristles attached to said handle portion proximate to said distal end thereof, the relatively stiff bristles being adapted for insertion into the patient's cervix to obtain endocervical cells and being centrally located relative to said flexible bristles and aligned along said axis.

2. A medical Pap smear brush as in claim 1 wherein the handle portion comprises two twisted wires which are twisted about each other.

3. A medical Pap smear brush as in claim 2 wherein the stiff bristles are held between the wires.

4. A medical Pap smear brush as in claim 1 wherein the handle portion is bendable and retains its bent shape when bent.

5. A medical Pap smear brush as in claim 1 wherein the flexible bristles are less than about 1 mm in diameter.

6. A medical Pap smear brush as in claim 1 wherein the stiff bristles extend generally (within 40°) perpendicular to said axis.

7. A medical Pap smear brush as in claim 1 wherein the flexible bristles have a free end forming a fan shape and the stiff bristles extend above said fan shape.
